(19) United States
(12) Patent Application Publication
(10) Pub. No.: US 2019/0357784 A1
(43) Pub. Date: Nov. 28, 2019

(54) BLOOD PRESSURE INFORMATION MEASUREMENT DEVICE CUFF

(71) Applicant: OMRON HEALTHCARE CO., LTD., Muko-shi (JP)

(72) Inventors: Chisato TAWARA, Kyoto (JP); Minoru TANIGUCHI, Kyoto (JP); Keita IKEDA, Kyoto (JP); Hirotaka HAYASHI, Kyoto (JP); Masaki HARADA, Kyoto (JP)

(73) Assignee: OMRON HEALTHCARE CO., LTD., Muko-shi (JP)

(21) Appl. No.: 16/535,904

(22) Filed: Aug. 8, 2019

Related U.S. Application Data
(63) Continuation of application No. PCT/JP2017/047055, filed on Dec. 27, 2017.

(30) Foreign Application Priority Data
Feb. 16, 2017 (JP) 2017-027191

(51) Int. Cl. A61B 5/022; A61B 5/0235; A61B 5/0225

(52) U.S. Cl. CPC A61B 5/02233; A61B 5/0225; A61B 5/02225; A61B 5/0235

(57) ABSTRACT

A blood pressure information measurement device cuff includes a band-like first fluid bag including a first nipple and a second fluid bag including a second nipple. The second fluid bag is accommodated in the first fluid bag, the second nipple is provided on a first main surface of a pair of main surfaces of the second fluid bag and extends outside through a main surface of a pair of main surfaces of the first fluid bag, the main surface facing the first main surface of the second fluid bag, and in a case that the first fluid bag and the second fluid bag are both unfolded flatly, the first nipple is disposed at a position corresponding to an outer edge section of the second fluid bag or a position further outward from the outer edge section.

### TECHNICAL FIELD

The present invention relates to a blood pressure information measurement device cuff including a fluid bag for compressing a living body and an outer cover for containing the fluid bag.

### BACKGROUND ART

Measuring blood pressure information is very important to know health state of a subject. Systolic blood pressure values (hereinafter referred to as systolic blood pressure), diastolic blood pressure values (hereinafter referred to as diastolic blood pressure), and the like are widely known as representative indicators in health management and known for their usefulness. In recent years, as well as systolic blood pressure and diastolic blood pressure, measuring pulse waves has also been used to find the cardiac load, degree of arteriosclerosis, and the like.

A blood pressure information measurement device is a device for obtaining indicators for health management based from the measured blood pressure information. There is a demand for such devices to have further application in fields such as the early detection, prevention, and treatment of circulatory system diseases. Note that the blood pressure information includes a broad range of information relating to the circulatory system and indicators such as the systolic blood pressure, the diastolic blood pressure, the mean blood pressure, the pulse wave, the pulse, and the degree of arteriosclerosis.

Generally, a blood pressure information measurement device cuff (hereinafter, also referred to simply as cuff) is used to measure blood pressure information. Here, “cuff” refers to a band-like or annular structure that includes a fluid bag with an empty space inside and is capable of being worn on a portion of the body, the cuff being used to measure blood pressure information via a gaseous or liquid fluid being inserted into the empty space to expand and contract the fluid bag. Note that, cuffs are also referred to as arm bands or manschettes.

Typically, the cuff is wrapped around a measurement site (e.g., the upper arm) in the length direction of the cuff. In a case where the length of the cuff in the width direction (length in the direction orthogonal to the length direction, i.e., cuff width) does not match the thickness of the measurement site, accurate blood pressure measurements may not be possible.

In the literature, for example, JP 2012-147995 A (Patent Document 1), a known blood pressure information measurement device determines a thickness of a measurement site and measures blood pressure information.

The blood pressure information measurement device described in Patent Document 1 includes a blood pressure information measurement device cuff that includes a first air bag and a second air bag contained in the first air bag. When using the blood pressure information measurement device to measure blood pressure information, the user enters in advance whether the measurement site is thick or thin. Then, the blood pressure information measurement device pressurizes the first air bag or the second air bag in accordance with the entered information. In a case where thick is entered, the first air bag is pressurized, and in a case where thin is entered, the second air bag is pressurized.

When the first air bag or the second air bag is pressurized, the time taken to reach a predetermined reference pressure (P1 when the first air bag is pressurized and P2 when the second air bag is pressurized) is measured. In a case where the time taken is less than a preset threshold (Th1 when the first air bag is pressurized and Th2 when the second air bag is pressurized), a determination unit determines the measurement site to be thin, and in a case where the time taken is longer than the preset threshold, the determination unit determines the measurement site to be thick.

In a case where the determination of the determination unit and the initially entered information match, the first air bag or the second air bag pressurized in accordance with the entered information is continuously pressurized and the blood pressure information is measured. In a case where the determination of the determination unit and the initially entered information do not match, the pressurization of the first air bag or the second air bag pressurized in accordance with the entered information is ceased, the other first air bag or second air bag is pressurized, and the blood pressure information is measured.

### CITATION LIST

### Patent Literature

Patent Document 1: JP 2012-147995 A

### SUMMARY OF INVENTION

### Technical Problem

As described above, in the blood pressure information measurement device cuff described in Patent Document 1, in a case where the user inputs information indicating that the measurement site is “thin” in advance, and the determination unit determines that the measurement site is “thick”, air is supplied to the first air bag after the second air bag is pressurized to the predetermined reference pressure.

However, in Patent Document 1, a first nipple provided on the first air bag and a second nipple provided on the second air bag are disposed coaxially to form a double tube structure. Therefore, depending on the reference pressure, the second air bag that is already inflated may block the first nipple portion from the inside, and thus air supplied to the first air bag may be hindered.

The present invention has been made in views of the problems described above. An object of the present invention is to provide a blood pressure information measurement device cuff, which has a configuration including a first fluid bag and a second fluid bag accommodated in the first fluid bag and can reliably supply fluid to both the first fluid bag and the second fluid bag.

### Solution to Problem

A blood pressure information measurement device cuff according to an embodiment of the present invention includes a first fluid bag having a band-like shape and including a first nipple configured to allow a fluid to enter and exit, the first fluid bag being configured to expand and contract by a fluid entering and exiting via the first nipple, and a second fluid bag having a band-like shape and including a second nipple configured to allow a fluid to enter and exit, the second fluid bag being configured to expand and contract by a fluid entering and exiting via the second nipple. The second fluid bag is accommodated in the first fluid bag, the second nipple is provided on a first main surface of a pair of main surfaces of the second fluid bag and extends outside through a main surface of a pair of main surfaces of the first fluid bag, the main surface facing the first main surface of the second fluid bag, and in a case that the first fluid bag and the second fluid bag are both unfolded flatly, the first nipple is disposed at a position corresponding to an outer edge section of the second fluid bag or a position further outside from the outer edge section.

In the blood pressure information measurement device cuff according to an embodiment of the present invention, the first fluid bag may include a length direction that corresponds to a circumferential direction in a case that the first fluid bag is wrapped at a measurement site and a width direction orthogonal to the length direction. In this case, when the first fluid bag and the second fluid bag are both unfolded flatly, the second fluid bag may be positioned at the center in the width direction of the first fluid bag.

In the blood pressure information measurement device cuff according to an embodiment of the present invention, the first fluid bag may include a length direction that corresponds to a circumferential direction in a case that the first fluid bag is wrapped at a measurement site and a width direction orthogonal to the length direction. In this case, when the first fluid bag and the second fluid bag are both unfolded flatly, the first nipple and the second nipple may be disposed along a direction parallel to the width direction.

### Advantageous Effects of Invention

The present invention can provide a blood pressure information measurement device cuff, which has a configuration including a first fluid bag and a second fluid bag accommodated in the first fluid bag and can reliably introduce fluid to both the first fluid bag and the second fluid bag.

### BRIEF DESCRIPTION OF DRAWINGS

FIG. 1 is a perspective view illustrating an external structure of a blood pressure monitor according to an embodiment.

FIG. 2 is a developed view of a first air bag and a second air bag according to the embodiment.

FIG. 3 is a cross-sectional view illustrating the first air bag and the second air bag illustrated in FIG. 2 in a pressurized state.

FIG. 4 is a functional block diagram illustrating the configuration of the blood pressure monitor according to the embodiment.

FIG. 5 is a flow chart illustrating the measurement flow of the blood pressure monitor according to the embodiment.

FIG. 6 is a perspective view illustrating an external structure of a blood pressure monitor according to a modified example.

### DESCRIPTION OF EMBODIMENTS

Embodiments of the present invention will be described in detail below with reference to the drawings. In the embodiments described below, a blood pressure cuff used in an upper arm blood pressure monitor that is capable of measuring blood pressure values including the systolic blood pressure and the diastolic blood pressure is used as an example of a blood pressure information measurement device cuff. Note that in the following, identical or common components are given the same reference signs in the drawings, and the descriptions thereof are not repeated.

### EMBODIMENTS

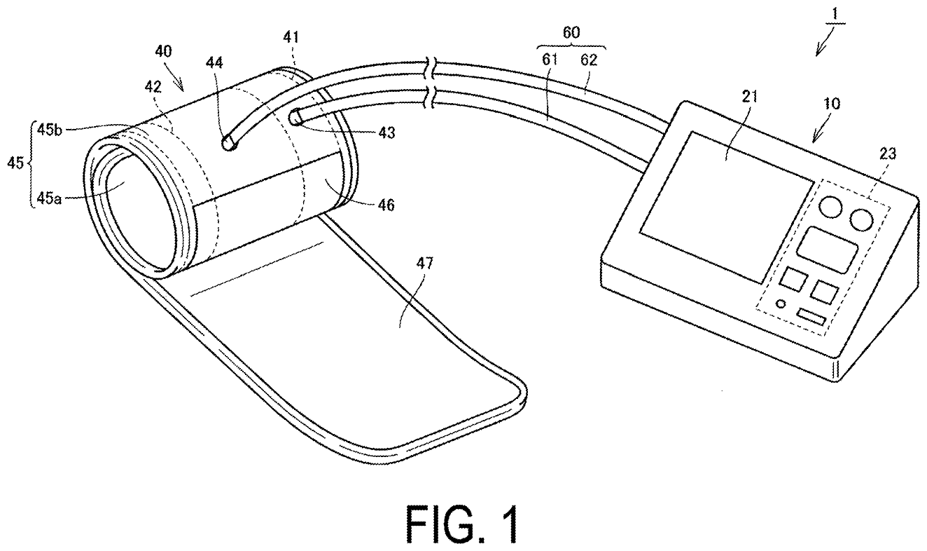

FIG. 1 is a perspective view illustrating an external structure of a blood pressure monitor according to an embodiment. A schematic configuration of a blood pressure monitor 1 according to the embodiment will be described with reference to FIG. 1.

As illustrated in FIG. 1, the blood pressure monitor 1 includes a body 10, a cuff 40, and an air tube 60 as a fluid supply path. The air tube 60 connects the separated body 10 and cuff 40.

The air tube 60 includes a first air tube 61 as a first supply path and a second air tube 62 as a second supply path. The first air tube 61 and the second air tube 62 are separated from each other, for example. The first air tube 61 and the second air tube 62 are each constituted by a flexible tube made of resin, for example.

The body 10 includes a box-like casing and includes a display unit 21 and an operation unit 23 on a top surface thereof. The body 10 is configured to be used placed on a placement surface such as a table when measurement is performed.

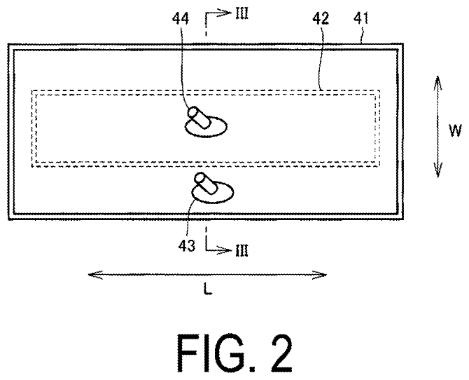

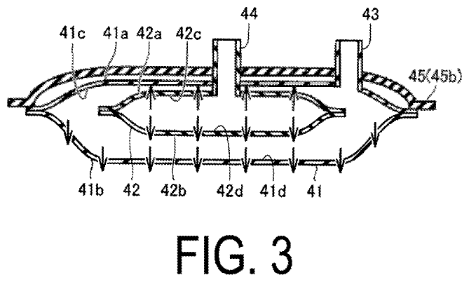

The cuff 40 has a band-like shape allowing it to be wrapped around the upper arm, i.e., the site where it is worn. The cuff 40 is used to be worn on the upper arm when measurement is performed. When wrapped around the upper arm in the worn state, the cuff 40 takes an annular form. The cuff 40 includes an outer cover 45, a first air bag 41 as a first fluid bag, and a second air bag 42 as a second fluid bag. Details of the first air bag 41 and the second air bag 42 will be described later using FIGS. 2 and 3.

The outer cover 45 has a bag-like shape and a band-like shape that is substantially rectangular in a plan view when the outer cover 45 is unfolded. The outer cover 45 includes an outer cover member 45b that is located radially outward when in a worn state, and an inner cover member 45a that is located radially inward and is in contact with the surface of the upper arm when in a worn state.

The outer cover 45 is formed in a bag-like shape by the outer cover member 45b and the inner cover member 45a being layered on one another, the edges being covered by a bias tape (not illustrated), and joined (for example, by sewing, welding, or the like).

A surface fastener 46 is provided on the outer circumferential surface of the outer cover 45 at/near a first end in the longitudinal direction, and a surface fastener 47 is provided on an inner circumferential surface of the outer cover 45 at/near a second end on the opposite side from the first end. The surface fastener 46 is constituted by a hook fastener, for example, and the surface fastener 47 is constituted by a loop fastener, for example.

The surface fasteners 46, 47 are configured to fasten together when the outer cover 45 is wrapped around the upper arm by the section of the outer cover 45 at/near the first end and the section of the outer cover 45 at/near the second end being layered on one another on the surface of the upper arm. Thus, by fastening together the surface fasteners 46, 47 with the cuff 40 wrapped around the upper arm, the outer cover 45 is fixed on the upper arm in the worn state.

FIG. 2 is a developed view of the first air bag and the second air bag according to the embodiment. FIG. 3 is a cross-sectional view illustrating the first air bag and the second air bag illustrated in FIG. 2 in a pressurized state. The first air bag 41 and the second air bag 42 will be described with reference to FIGS. 2 and 3.

As illustrated in FIG. 2, the first air bag 41 has a bag-like shape and a band-like shape that is substantially rectangular in a plan view when the first air bag 41 is unfolded. The first air bag 41 includes a length direction L that corresponds to the circumferential direction when the first air bag 41 is wrapped at the measurement site and a width direction W orthogonal to the length direction L.

The first air bag 41 includes a pair of outer surfaces 41a, 41b and a pair of inner surfaces 41c, 41d.

A first nipple 43 is provided on the outer surface 41a of the pair of outer surfaces 41a, 41b. The first air bag 41 expands and contracts by air entering and exiting via the first nipple 43.

The second air bag 42 has a bag-like shape and a band-like shape that is substantially rectangular in a plan view when the second air bag 42 is unfolded. The outer shape of the second air bag 42 is smaller than the outer shape of the first air bag 41. The second air bag 42 is accommodated in the first air bag 41. The second air bag 42 is centrally positioned in the width direction W of the first air bag 41 in a state in which the first air bag 41 and the second air bag 42 are both unfolded flatly. Additionally, the second air bag 42 is centrally positioned in the length direction L of the first air bag 41 in a state in which the first air bag 41 and the second air bag 42 are both unfolded flatly.

The second air bag 42 includes a pair of outer surfaces 42a, 42b and a pair of inner surfaces 42c, 42d. The pair of outer surfaces 42a, 42b of the second air bag 42 are disposed facing the pair of inner surfaces 41c, 41d of the first air bag 41.

A second nipple 44 is provided on the outer surface 42a of the pair of outer surfaces 42a, 42b. The second air bag 42 expands and contracts by air entering and exiting via the second nipple 44.

The second nipple 44 is roughly centrally disposed in the longitudinal direction and the width direction of the second air bag 42, for example. By disposing the second nipple 44 in such a position, the second air bag 42 can be expanded in a roughly uniform manner.

When the first air bag 41 and the second air bag 42 are unfolded flatly, the first nipple 43 is preferably disposed at a position corresponding to the outer edge section of the second air bag 42 or a position further outward than the outer edge section.

Here, a position corresponding to the outer edge section of the second air bag 42 or a position further outward than the outer edge section is a position such that the first nipple 43 is not blocked by the second air bag 42 in the worn state, i.e., when the first air bag 41 is not expanded and the second air bag 42 is expanded. Accordingly, “a position corresponding to the outer edge section of the second air bag 42” includes a position not just overlapping with the outer edge section of the second air bag 42, but also a position a certain amount inward from the outer edge section of the second air bag 42 in a plan view when the first air bag 41 and the second air bag 42 are unfolded.

Also, when both the first air bag 41 and the second air bag 42 are unfolded flatly, the first nipple 43 is preferably disposed side by side with the second nipple 44 in a direction parallel with the width direction of the first air bag 41.

In the embodiment, when both the first air bag 41 and the second air bag 42 are unfolded flatly, the first nipple 43 is disposed at a position outward from the outer edge section of the second air bag, side by side with the second nipple 44 in a direction parallel with the width direction of the first air bag 41. By this configuration, the first air tube 61 and the second air tube 62 can be disposed closely to each other and thus an unbulky structure can be achieved.

The second nipple 44 extends outside through the inner surface 41c of the first air bag 41 disposed facing the outer surface 42a of the second air bag 42.

The first air bag 41 and the second air bag 42 are each preferably constituted by a bag-like member formed using a resin sheet. The material of the resin sheet constituting the first air bag 41 and the second air bag 42 can be any material that is highly elastic and can prevent air from leaking from the internal space. From this perspective, suitable materials for the resin sheet include ethylene-vinyl acetate copolymers, soft vinyl chloride, polyurethane, and polyamide.

As illustrated in FIG. 3, when blood pressure is measured, the first air bag 41 and the second air bag 42 are pressurized and expanded. Additionally, the blood pressure is preferably measured in a state in which internal pressure of the second air bag 42 is greater than that of the first air bag 41. The first air bag 41 may be pressurized first or the second air bag 42 may be pressurized first.

As described above, when the first air bag 41 and the second air bag 42 are unfolded flatly, the first nipple 43 is disposed at a position corresponding to the outer edge section of the second air bag 42 or a position further outward than the outer edge section. Thus, in the case of the second air bag 42 being filled with air and expanded before the first air bag 41, the first nipple 43 can be prevented from being blocked by the second air bag 42. This allows air to be reliably introduced inside the first nipple 43.

Additionally, in a case that the internal pressure of the second air bag 42 is greater than the internal pressure of the first air bag 41, in accordance with Pascal's principle, even in a case of a small amount of air being supplied to the first air bag 41, the force of the first air bag 41 pressing against the measurement site can be amplified.

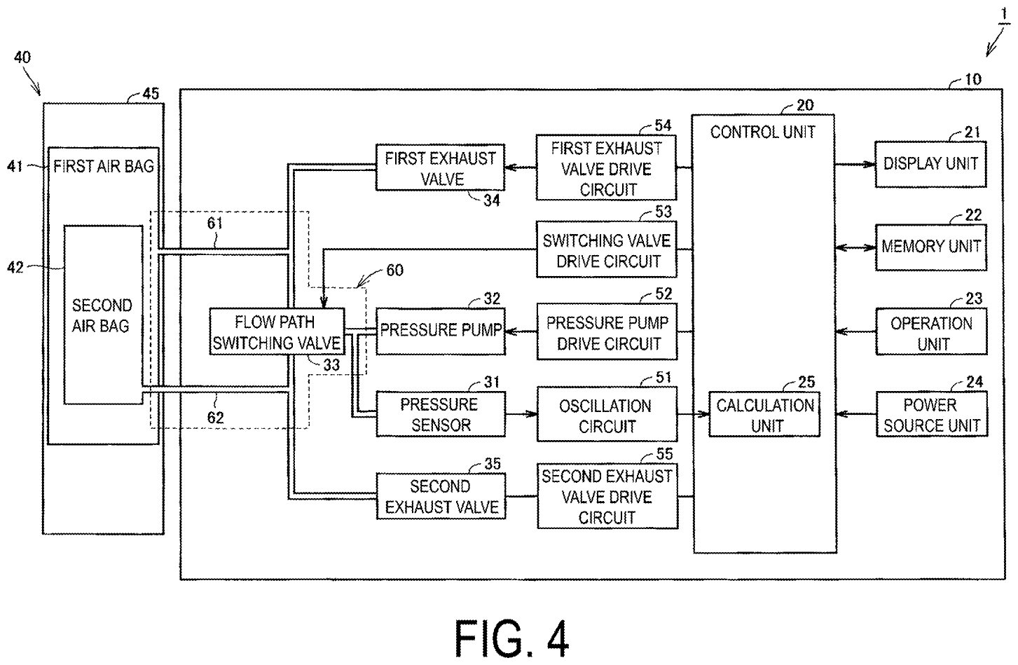

FIG. 4 is a functional block diagram illustrating the configuration of the blood pressure monitor according to the embodiment. The functional blocks of the blood pressure monitor 1 will be described with reference to FIG. 4.

As illustrated in FIG. 4, in addition to the display unit 21 and the operation unit 23 described above, the body 10 includes a control unit 20, a memory unit 22, a power source unit 24, a pressure sensor 31, a pressure pump 32, a flow path switching valve 33, a first exhaust valve 34, a second exhaust valve 35, an oscillation circuit 51, a pressure pump drive circuit 52, a switching valve drive circuit 53, a first exhaust valve drive circuit 54, and a second exhaust valve drive circuit 55.

The pressure pump 32, the first exhaust valve 34, the flow path switching valve 33, and the second exhaust valve 35 correspond to a pressure increase/reduction mechanism that increases or decreases the pressure in the internal space of the first air bag 41 and the second air bag 42.

The pressure increase/reduction mechanism is configured to be switchable from a first state in which one fluid bag of the first air bag 41 and the second air bag 42 is pressurized and expanded, to a second state in which, while a sealed state of one fluid bag of the fluid bags is maintained, the other fluid bag of the first air bag 41 and the second air bag 42 is pressurized and expanded.

The control unit 20 is constituted by a central processing unit (CPU) and, for example, is configured to comprehensively control the blood pressure monitor 1. The control unit 20 includes a calculation unit 25 that calculates the blood pressure on the basis of the pressure information detected by the pressure sensor 31.

The memory unit 22 is constituted by read-only memory (ROM) and random-access memory (RAM) and, for example, is configured to store a program for causing the control unit 20 and the like to execute a processing procedure for measuring blood pressure values and store measurement results and the like.

The display unit 21 is constituted by a liquid crystal display (LCD) and, for example, is configured to display measurement results and the like. The operation unit 23 is configured to receive an operation by a user or the like and allow the instruction from the outside to be input into the control unit 20 and the power source unit 24. The power source unit 24 is configured to supply power to the control unit 20.

The control unit 20 inputs control signals for driving the pressure pump 32, the flow path switching valve 33, the first exhaust valve 34, and the second exhaust valve 35 into the pressure pump drive circuit 52, the switching valve drive circuit 53, the first exhaust valve drive circuit 54, and the second exhaust valve drive circuit 55, respectively. Additionally, the control unit 20 inputs the blood pressure value calculated by the calculation unit 25 into the memory unit 22 and the display unit 21 as a measurement result.

Note that the blood pressure monitor 1 may separately include an output unit configured to output the blood pressure value as a measurement result to an external device (for example, a personal computer (PC), a printer, or the like). For example, a serial communication line, a writing device that writes the blood pressure value to various types of recording medium, or the like can be used as the output unit.

The pressure pump 32 increases the internal pressure of the first air bag 41 and the internal pressure of the second air bag 42 by supplying air to the internal space of the first air bag 41 and the second air bag 42. The pressure pump 32 supplies air to the first air bag 41 and the second air bag 42 via the air tube 60. An end on a first side of the air tube 60 is connected to the pressure pump 32. An end on a second side of the air tube 60 branches into the first air tube 61 connected to the first air bag 41 and the second air tube 62 connected to the second air bag 42.

The distal end of the first air tube 61 is inserted into the first nipple 43 and connected to the first air bag 41. The distal end of the second air tube 62 is inserted into the second nipple 44 and connected to the second air bag 42.

The pressure pump drive circuit 52 controls the operation of the pressure pump 32 on the basis of a control signal received from the control unit 20.

The flow path switching valve 33 is provided at a point along the air tube 60. Specifically, the flow path switching valve 33 is provided at a section that branches into the first air tube 61 and the second air tube 62. The flow path switching valve 33 switches between a state in which air is supplied to the first air bag 41 via the first air tube 61 (a state in which the first air bag 41 is pressurized) and a state in which air is supplied to the second air bag 42 via the second air tube 62 (a state in which the second air bag 42 is pressurized).

The switching valve drive circuit 53 controls the operation of the flow path switching valve 33 on the basis of a control signal received from the control unit 20.

The first exhaust valve 34 is connected to the first air tube 61. By opening/closing the first exhaust valve 34, the internal pressure of the first air bag 41 is maintained or the internal space of the first air bag 41 is opened to the outside to reduce the internal pressure of the first air bag 41.

The first exhaust valve drive circuit 54 controls the operation of the first exhaust valve 34 on the basis of a control signal received from the control unit 20.

The second exhaust valve 35 is connected to the second air tube 62. By opening/closing the second exhaust valve 35, the internal pressure of the second air bag 42 is maintained or the internal space of the second air bag 42 is opened to the outside to reduce the internal pressure of the second air bag 42.

The second exhaust valve drive circuit 55 controls the operation of the second exhaust valve 35 on the basis of a control signal received from the control unit 20.

The internal pressure of the first air bag 41 or the internal pressure of the second air bag 42 can be measured using the pressure sensor 31. When the first air tube 61 and the pressure pump 32 are communicated with each other by the flow path switching valve 33, the internal pressure of the first air bag 41 can be measured. When the second air tube 62 and the pressure pump 32 are communicated with each other by the flow path switching valve 33, the internal pressure of the second air bag 42 can be measured.

The pressure sensor 31 is a capacitive sensor. The electrostatic capacitance of the pressure sensor 31 varies depending on the internal pressure of the first air bag 41 or the internal pressure of the second air bag 42. The oscillation circuit 51 generates a signal having an oscillation frequency in accordance with the electrostatic capacitance of the pressure sensor 31 and inputs the generated signal to the control unit 20.

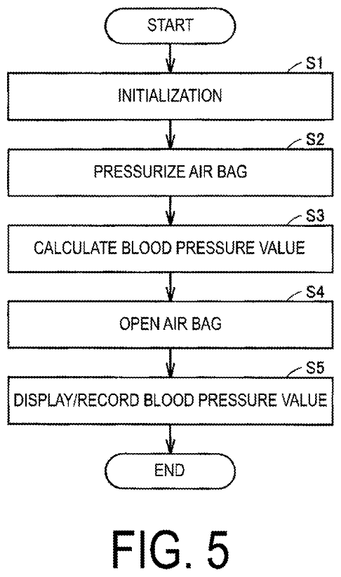

FIG. 5 is a flow chart illustrating a measurement flow of the blood pressure monitor according to the embodiment. With reference to FIG. 5, the measurement flow of the blood pressure monitor 1 will be described.

When measuring blood pressure values, the cuff 40 is wrapped around the upper arm of the subject and worn in advance. In this state, when the operation unit 23 provided in the body 10 is operated and the blood pressure monitor 1 is turned on, power is supplied to the control unit 20 from the power source unit 24 to drive the control unit 20.

As illustrated in FIG. 5, after the control unit 20 is driven, the control unit 20 first initializes the blood pressure monitor 1 (step S1). In the initialization, the control unit 20 causes the first air bag 41 and the second air bag 42 to be in an open state in which the internal space of the first air bag 41 and the second air bag 42 is opened to the outside by controlling the operation of the first exhaust valve 34 and the second exhaust valve 35.

Next, the control unit 20 waits for an instruction to start the measurement, and when the operation unit 23 is operated and an instruction to start the measurement is input, the control unit 20 causes the first exhaust valve 34 and the second exhaust valve 35 to be closed and starts driving the pressure pump 32 (step S2).

In step S2, the control unit 20 controls operation of the flow path switching valve 33 such that air is supplied to the first air bag 41 or the second air bag 42. When the pressure of the first air bag 41 or the second air bag 42 reaches a predetermined pressure, the control unit 20 controls the operation of the flow path switching valve 33 such that air is supplied to the other first air bag 41 or second air bag 42.

Note that the predetermined pressure is a reference pressure P1 in the case of pressurizing the first air bag 41 first, and a reference pressure P2 in the case of pressurizing the second air bag 42 first. Here, the reference pressure P1 is less than the reference pressure P2.

As the air is supplied to the other first air bag 41 or second air bag 42, the internal pressure of both the first air bag 41 and the second air bag 42 is increased.

In the process of pressurizing, the control unit 20 calculates the systolic blood pressure and the diastolic blood pressure using a known procedure (step S3). Specifically, in the process of increasing the internal pressure of both the first air bag 41 and the second air bag 42, the control unit 20 obtains the internal pressure of the first air bag 41 from the oscillation frequency obtained from the oscillation circuit 51 and extracts the pulse wave information superimposed on the obtained internal pressure of the first air bag 41. Then, the control unit 20 calculates the blood pressure value on the basis of the extracted pulse wave information.

When the blood pressure value is calculated in step S3, the control unit 20 stops driving the pressure pump 32 and opens the first exhaust valve 34 and the second exhaust valve 35 to completely exhaust the air in the first air bag 41 and the second air bag 42 (step S4).

Additionally, the blood pressure value is displayed on the display unit 21 as the measurement result, and the blood pressure value is stored in the memory unit 22 (step S5).

Thereafter, the control unit 20 waits for an instruction to power off, and when the operation unit 23 is operated and an instruction to power off is input, the supply of power from the power source unit 24 to the control unit 20 is stopped, and the sequence of processing procedures is ended.

As described above, the cuff 40 according to the embodiment has a configuration including the first air bag 41 including the first nipple 43 and the second air bag 42 accommodated in the first air bag 41 and including the second nipple 44 penetrating the first air bag 41 in the protruding direction of the first nipple 43, as described above. Further, when the first air bag 41 and the second air bag 42 are both unfolded flatly, the first nipple 43 is disposed at a position corresponding to the outer edge section of the second air bag 42 or a position further outward from the outer edge section.

The first nipple 43 is disposed in the above-described manner. This can prevent the first nipple 43 from being blocked by the second air bag 42, even in a case that, in step 2 in the measurement flow, air is supplied to the first air bag 41 after air is supplied to the second air bag 42, and the second air bag 42 is filled with air and expanded before the first air bag 41. This allows air to be reliably supplied to the first air bag 41 via the first nipple 43.

Note that when the first air bag 41 is expanded before the second air bag 42 is expanded, the second nipple 44 is not blocked by the first air bag 41, and thus, as a matter of course, air can be reliably supplied to the second air bag 42 via the second nipple 44.

As described above, in the cuff 40 according to the embodiment having a configuration including the first air bag 41 and the second air bag 42 accommodated in the first air bag 41, the fluid can be reliably supplied to both the first air bag 41 and the second air bag 42. Thus, blood pressure information can be stably measured.

### Modified Examples

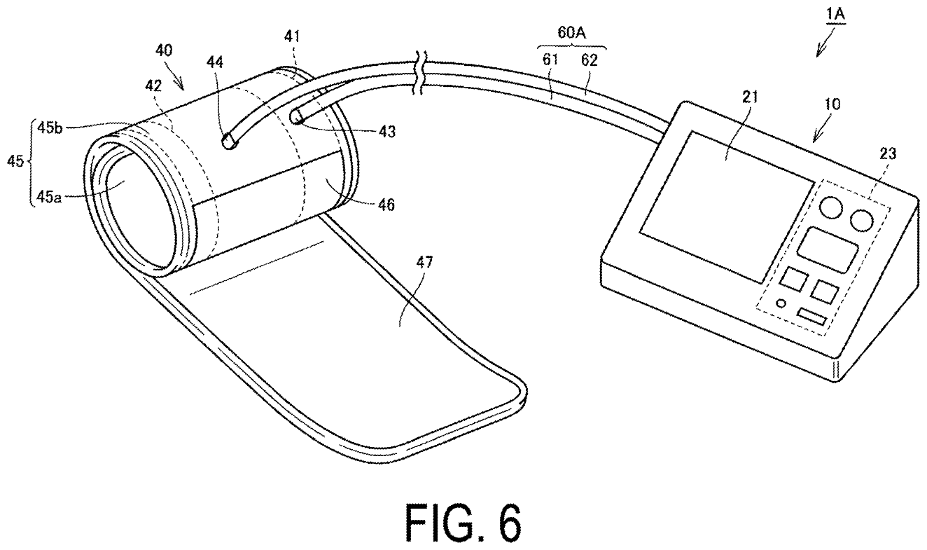

FIG. 6 is a perspective view illustrating an external structure of a blood pressure monitor according to a modified example. The external structure of the blood pressure monitor according to the modified example will be described with reference to FIG. 6.

As illustrated in FIG. 6, a blood pressure monitor 1A according to the modified example differs from the blood pressure monitor 1 according to the embodiment in that the configuration of an air tube 60A is different. Other configurations are substantially similar. The first air tube 61 and the second air tube 62 of the air tube 60A are formed together on the body 10 side. Specifically, on the body 10 side, a trunk section of the first air tube 61 and a trunk section of the second air tube 62 are connected, and the first air tube 61 and the second air tube 62 have a multi-trunk structure. On the first nipple 43 and the second nipple 44 side, the first air tube 61 and the second air tube 62 are branched off from one another. The air tube 60A may be configured in this manner. In the blood pressure monitor 1A according to the modified example, blood pressure can be measured via a measurement flow substantially similar to that of the embodiment.

In the embodiment and the modified example described above, the measurement method is based on the pressure measurement method. However, of course, it is also possible to employ a so-called pressure reduction measurement method in which the pulse waves are detected when the pressure of the first air bag 41 and the second air bag 42 is decreased.

In addition, in the embodiment and the modified example described above, a case in which the single pressure pump 32 is used and the destination of the air supply is switched between the first air bag 41 and the second air bag 42 by the flow path switching valve 33 is described as an example, but no such limitation is intended. Thus, a first pressure pump for pressurizing the first air bag 41 and a second pressure pump for pressurizing the second air bag 42 may be provided independently. In this case, the first pressure pump and the first air bag 41 are connected by the first air tube, and the second pressure pump and the second air bag 42 are connected by the second air tube. Each of the first air tube and the second air tube includes an independent flow path.

Additionally, in the embodiment and modified example described above, a case is described, as an example, in which the pressure sensor 31 is used to measure the internal pressure of the first air bag 41 or the second air bag 42, but no such limitation is intended. Thus, a first pressure sensor for measuring the internal pressure of the first air bag 41 and a second pressure sensor for measuring the internal pressure of the second air bag 42 may be provided independently.

As described above, the configuration of the body 10 in the embodiment and the modified example described above may be modified as appropriate, and thus, it is possible to employ a configuration in which the measurement is performed in a state in which a difference in pressure between the pressure of the first air bag 41 and the pressure of the second air bag 42 is kept constant. For example, a differential pressure valve may be used in place of the flow path switching valve 33, or a difference in pressure between the pressure of the first air bag 41 and the pressure of the second air bag 42 may be kept constant by controlling the operation of the above-described first pressure pump and second pressure pump.

Furthermore, in the embodiment and the modified example described above, a case is described, as an example, in which the measurement is performed in a state in which both the first air bag 41 and the second air bag 42 are pressurized, but no such limitation is intended. Thus, the measurement may be performed in a state in which only one of the first air bag 41 and the second air bag 42 is pressurized.

In the embodiment and the modified example described above, an air bag in which air can enter and exit has been used as the fluid bag, but no such limitation is intended. A bag in which a gas other than air or a non-compressible viscous fluid other than air enters and exits can also be used. In other words, in the embodiment described above, compressed air is used as the flow-controlled fluid, but application of the contents described above is not limited thereto. A high-pressure gas other than compressed air, a liquid in a compressed environment, or the like may be used as the flow-controlled fluid.

Embodiments of the present invention have been described above, but the embodiments described herein are illustrative in all respects and are not intended as limitations. The scope of the present invention is indicated by the claims and includes all meaning equivalent to the scope and changes within the scope.

### REFERENCE SIGNS LIST

1, 1A Blood pressure monitor
10 Body
20 Control unit
21 Display unit
22 Memory unit
23 Operation unit
24 Power source unit
25 Calculation unit
31 Pressure sensor
32 Pressure pump
33 Flow path switching valve
34 First exhaust valve
35 Second exhaust valve
40 Cuff
41 First air bag
41a, 41b Outer surface
41c, 41d Inner surface
42 Second air bag
42a, 42b Outer surface
42c, 42d Inner surface
43 First nipple
44 Second nipple
45 Outer cover
45a Inner cover member
45b Outer cover member
46, 47 Surface fastener
51 Oscillation circuit
52 Pressure pump drive circuit
53 Switching valve drive circuit
54 First exhaust valve drive circuit
55 Second exhaust valve drive circuit
60, 60A Air tube
61 First air tube
62 Second air tube

1. A blood pressure information measurement device cuff, comprising:
a first fluid bag having a band-like shape and comprising a first nipple configured to allow a fluid to enter and exit, the first fluid bag being configured to expand and contract by a fluid entering and exiting via the first nipple; and
a second fluid bag having a band-like shape and comprising a second nipple configured to allow a fluid to enter and exit, the second fluid bag being configured to expand and contact by a fluid entering and exiting via the second nipple, wherein
the second fluid bag is accommodated in the first fluid bag,
the second nipple is provided on a first main surface of a pair of main surfaces of the second fluid bag and extends outside through a main surface of a pair of main surfaces of the first fluid bag, the main surface facing the first main surface of the second fluid bag, and
in a case that the first fluid bag and the second fluid bag are both unfolded flatly, the first nipple is disposed at a position corresponding to an outer edge section of the second fluid bag or a position further outward from the outer edge section.

2. The blood pressure information measurement device cuff according to claim 1, wherein
the first fluid bag comprises a length direction that corresponds to a circumferential direction in a case that the first fluid bag is wrapped at a measurement site and a width direction orthogonal to the length direction, and
in a case that the first fluid bag and the second fluid bag are both unfolded flatly, the second fluid bag is positioned at the center in the width direction of the first fluid bag.

3. The blood pressure information measurement device cuff according to claim 1, wherein
the first fluid bag comprises a length direction that corresponds to a circumferential direction in a case that the first fluid bag is wrapped at a measurement site and a width direction orthogonal to the length direction, and
in a case that the first fluid bag and the second fluid bag are both unfolded flatly, the first nipple and the second nipple are disposed along a direction parallel to the width direction.

4. The blood pressure information measurement device cuff according to claim 2, wherein
the first fluid bag comprises a length direction that corresponds to a circumferential direction in a case that the first fluid bag is wrapped at a measurement site and a width direction orthogonal to the length direction, and
in a case that the first fluid bag and the second fluid bag are both unfolded flatly, the first nipple and the second nipple are disposed along a direction parallel to the width direction.
